(19) United States
(12) Patent Application Publication
(10) Pub. No.: US 2009/0209931 A1
(43) Pub. Date: Aug. 20, 2009

(54) Method for the Production of a Plurality of Elastic Disposable incontinence Diapers Absorbing Body Fluids

(76) Inventor: Thomas Wurster, Heidenheim (DE)

Correspondence Address: DREISS, FUHLENDORF, STEIMLE & BECKER, POSTFACH 10 37 62, D-70032 STUTTGART, DE

(21) Appl. No.: 11/921,458

(22) PCT Filed: Jul. 6, 2006
(86) PCT No.: PCT/EP2006/006585

§ 371 Date: Dec. 3, 2007

(30) Foreign Application Priority Data
Jul. 9, 2005 (DE) 10 2005 032 221.2

(51) Int. Cl. A61F 13/49; D04B 11/26

(52) U.S. Cl. 604/367; 28/153

(57) ABSTRACT

The invention relates to a method for producing a plurality of elastic disposable incontinence diapers (200) which absorb body fluids and are provided in the form of pants. The method comprises the following steps: an elastic web of material (4) that is continuous in a longitudinal direction (2) and is delimited by a first and a second longitudinal edge (16,18) is produced by means of a textile process during which openings (6) are formed successively in the longitudinal direction (2) in the web of material (4) at a predefined distance from each other, the web of material (4) is fed in the longitudinal direction (2); absorbing elements (10) are supplied and delivered; said absorbing elements (10) are successively applied to the web of material (4), and one respective absorbing elements (10) is fixed between two openings (6) on the web of material (4); the web of material (4) is folded over about an axis (20) extending in the longitudinal direction (2); the folded-over web of material (4) is fixed along a direction (24) that runs perpendicular to the longitudinal direction (2); the disposable incontinence diapers (200) are separated by cutting the folded-over web of material (4) perpendicular to the longitudinal direction in predetermined sections.

The invention relates to a method for the manufacture of a multiplicity of elastic disposable incontinence diapers in closed form (pants form often also called disposable diapers) which absorb body fluids.

Disposable incontinence diapers in closed form are currently conventional manufactured in a continuous manner using non-woven fabric webs, with the non-woven fabric webs being provided with an elastifiable material, as a rule in form of elastic threads. During the course of the continuous manufacture, sections are punched out of the non-woven fabric webs in order to form the leg apertures of the disposable diaper. The non-woven fabric web is then folded onto itself or non-woven fabric webs are fed on top of each other and joined with each other in sections to form the lateral seams of the disposable diaper. This can take place before or after separation from the continuous web. As an example, reference is made to EP 0 048 011 B1 and EP 0 405 575 A1 as prior art.

With the known methods, additional means must disadvantageously be provided for elastification of the flat material of the non-woven fabric web and joined with the flat material in a suitable manner. In addition, it is also disadvantageous that openings for the leg apertures of the disposable diaper being manufactured must be punched out. This involves cut waste, which is problematic during the course of the continuous manufacture since the waste must be removed from the manufacturing machine and disposed of. In addition, this cut waste also constitutes unusable material consumption and thus a disadvantageous cost factor.

DE 80 03 143 U1, DE 30 04 469 C2 and DE 37 40 845 C1 describe woven fishnet panties and methods for their manufacture.

It is the object of the present invention to develop a method of the kind mentioned above that can be carried out in an economical manner and with which the aforementioned disadvantages do not occur.

According to the invention, this object is solved by a method with the features of claim 1.

For the material web forming the outer envelope of the disposable incontinence diaper, a textile material is more preferably selected which is elastic in a longitudinal direction (corresponding to the manufacturing direction). Advantageously, the textile material is also elastic in a transverse direction, i.e. transverse to the manufacturing direction. Particularly advantageous is a material whose elastic characteristics differ in the longitudinal and transverse directions, in particular with regard to the force of the material counteracting elongation. More preferably, the force of the material counteracting elongation can be less in the longitudinal direction than in the transverse direction.

A textile method for the manufacture of the elastic material web in accordance with the invention is hereby defined as a method with which textile bonds are formed using threads, i.e. a weaving method, knitting method or methods of such kind. This has the advantage that, with the embodiment of this textile method, apertures can be formed in the material web which later form the leg apertures of the disposable diaper, in particular, without resulting in cut waste that would have to be removed and disposed of and which constitutes a disadvantageous cost factor.

Since the absorbent body is placed on the unfolded flat material web, it can also be easily joined with the flat material web during manufacture and, if desired, evenly over its entire extension such that, in later use of the hygiene article, it does not change its intended position, even if forces are exerted on the absorbent body as a result of user body movements which twist the absorbent body joined to the material web. If desired, the manufacture of an adequately resistant bond between the absorbent body and the material web can be supported through suitable method measures such as the pressing-on with roller pairs. Only after an adequate joint between absorbent body and material web is achieved within the manufacturing machine is the material web folded onto itself and fixed onto or with itself for forming the lateral seam regions of the disposable diaper, and then separated.

According to a version of the method according to the invention, the openings forming the leg apertures of a disposable incontinence diaper are provided in the longitudinal direction of the material web, one after the other. In other words, a single row of apertures extending in longitudinal direction is formed in the material web. The disposable incontinence diapers to be manufactured are thus conveyed in the manufacturing machine in their subsequent transverse direction (transverse conveying).

According to another version, the apertures forming the leg apertures of a disposable incontinence diaper are provided next to each other in pairs transverse to the longitudinal direction of the material web. This means that, when viewing the material web from the top, two rows of successive apertures which extend in the longitudinal direction and which are oriented parallel to each other are formed, wherein the apertures belonging to a respective disposable incontinence diaper are arranged next to each other transverse to the longitudinal direction. The disposable incontinence diapers are thus conveyed in their longitudinal direction (longitudinal conveying).

With the method version mentioned above, the usually oblong absorbent bodies are consequently arranged on the material web with their longitudinal extension transverse to the longitudinal direction or production direction of the method. With the first method version, the absorbent bodies are each advantageously arranged between groups of two consecutive apertures in the longitudinal direction. In this manner, each aperture forms a leg aperture in the material web. In prior art, the leg apertures were formed through punching of non-woven fabric materials, the subsequent severing cut for separating the hygiene article being effected through a respective punching, so that a respective punching was responsible for forming two leg apertures of articles abutting each other. However, this reduced the height or extension of the lateral seam section of the disposable diaper. This is now no longer the case. It may nevertheless prove desirable if an absorbent body is disposed between all openings in the material web which form leg apertures. With the manufacture in transverse conveyance, an alternating sequence of aperture and absorbent body then results, in the manufacturing direction. The severing cut for separating the articles must however pass through a leg aperture in such a case.

With the second method version (longitudinal conveyance) each absorbent body is positioned between two apertures provided next to each other transverse to the longitudinal direction, and fixed in this position.

Positioning and fixing of the absorbent body on the still continuous material web can take place immediately following manufacture of the material web in both method versions, as a single integrated manufacturing method. However, separation of these method steps is also conceivable and advantageous, more preferably such that, following manufacture of the material web with the apertures, the material web is initially temporarily stored, in particular, wound-up into a continuous roll. In a downstream process, the material web can again be fed continuously and positioning of the absorbent bodies can be carried out.

Non-woven materials, in particular, absorbent non-woven materials and/or super-absorbent polymer materials, are advantageously used for manufacturing the absorbent bodies. It also proves advantageous if an absorption core is used for the absorbent body, which is overlaid by a liquid-permeable top sheet and backed by a liquid-impermeable back sheet, i.e. arranged between the top and back sheets. In this manner, a visually attractive absorbent body with a soft feel can be created with which even materials, e.g. super-absorbent polymer materials, which are loosely contained in the absorbent body, can be held within the absorbent body and do not penetrate towards the outside. In addition, a liquid seal is achieved by the liquid-impermeable back sheet. The back sheet advantageously extends completely around the absorbent body and preferably forms an upwardly projecting receptacle for the absorption core. In a further embodiment according to the invention, the top and back sheets are joined to each other at least by sections outside the absorption core more preferably in a liquid-tight manner in order to form a receptacle for the intermediate absorption core.

In a further advantageous design of the invention, upright band elements are provided on both sides of the absorbent body, extending in the longitudinal direction of the absorbent body to effect lateral leak protection. These band elements are preferentially elastified, more preferably provided with thread-shaped elastification means, which lift the band elements against the body of the user wearing the disposable incontinence diaper. In this way, sideward barriers for solid and liquid body excretions are formed.

As mentioned above, within the course of carrying-out the method according to the invention, the absorbent body can advantageously be securely fastened to the material web. According to a version of the invention, the absorbent body is joined with the material web throughout its entire seating area. Joining can take place thermally via a continuous or discontinuous joining pattern. More preferably, joining can take place by way of an adhesive, more preferably a hot-melt adhesive. The hot-melt adhesive can be arranged over the full area or preferably in form of an open pattern, for example as a grid or spirals overlapping one another.

According to another method version, the absorbent body is only fixed to the material web through an area of a maximum of 95%, more preferably of a maximum of 90%, and furthermore more preferably of a maximum of 80%. The previously mentioned joining methods and joining patterns are again advantageously used. According to this method version, only a central region of the side of the absorbent body facing away from the body is advantageously fixed to the material web. In a particularly advantageous embodiment, the lateral longitudinal edge regions and/or the transverse edge regions of the absorbent body remain unfixed. When using an adhesive joining method, this has the advantage that unused adhesive application, which extends beyond the edges of the absorbent body and interferes with the further processing, can be safely avoided. The unfixed edges of the absorbent body have an extension, measured as shortest distance from the absorbent body edge to the start of the fixation, of more preferably at least 3 mm, furthermore more preferably of at least 8 mm, furthermore more preferably of at least 15 mm.

At least 30% by weight, more preferably a maximum of 90% by weight, more preferably a maximum of 80% by weight and further more preferably a maximum of 70% by weight of super-absorbent polymer materials (SAP) based on the mass of the absorption core are advantageously used for manufacture of the absorbent body.

It can prove advantageous that the axis which lies in the longitudinal direction, about which the material web is folded onto itself, is oriented along a longitudinal center line of the material web, i.e. has an equal distance from the first and the second longitudinal edges.

Although there is no fundamental restriction for selection of the materials for the manufacture of the textile material web, cotton is advantageously used for this purpose, i.e. threads of or at least with cotton. However, it would also be conceivable and advantageous for certain applications if a thermoplastic material is used in manufacture of the material web, i.e. threads of or with a thermoplastic material. In this case, a joint can be established through use of heat and/or pressure.

Fixation of the material web folded onto itself is advantageously carried out through thermal fusion or ultrasonic fusion along a direction transverse to the longitudinal direction.

An adhesion-promoting binder is advantageously used for fixing the material web folded onto itself, that is during formation of the joining points of the disposable diaper which normally form lateral seam regions. This can be a heat-meltable thermoplastic material or an adhesive, more preferably a hot melt adhesive, or a strip or thread-shaped section which is capable of forming adhesion-promotion between regions of the material web folded onto one another, more preferably when exerting heat and/or pressure.

According to a further version, a lateral seam can be formed along a direction transverse to the longitudinal direction for fixation of the material web folded onto itself using at least one additional thread.

According to a further inventive idea, the material web fixed onto itself is advantageously capable of being torn open again along this fixation. According to yet another inventive idea, the material web fixed onto itself is advantageously capable of being opened and closed again along the fixation.

A breathable yet liquid-impermeable back sheet is advantageously used for manufacture of the absorbent body. The breathability of the back sheet in such a case amounts to at least 300 g/m²/24 h, more preferably at least 1000 g/m²/24 h, further more preferably at least 2000 g/m²/24 h, further more preferably at least 3000 g/m²/24 h, further more preferably at least 4000 g/m²/24 h, further more preferably a maximum of 6000 g/m²/24 h measured according to DIN 53 122-1 (Edition: 2001-08).

Regardless of this, an elastic back sheet is advantageously used in manufacturing the absorbent body.

Furthermore, the elastic material web is advantageously formed with a breathability that is at least twice the breathability, more preferably at least three times the breathability of that of the absorbent body back sheet.

Following separation and depending on the folding of the material web, the disposable incontinence diapers are advantageously turned inside out using a reversing device so that they can be manufactured with the absorbent body located on the inside and passed on to the end user packed in this form.

If the disposable incontinence diapers are manufactured using transverse conveyance, the apertures can be formed along a longitudinal center line with the same distance to the first and the second longitudinal edges or, according to another version, they can be formed off-center, i.e. closer to one of the two longitudinal edges.

The feeding of the material web at the time of positioning of the absorbent body advantageously takes place at a web speed of at least 50 m/min, more preferably of at least 100 m/min.

According to a further advantageous inventive idea, the material web is fed to the manufacturing machine in a stretched state, and the stretch more preferably amounts to a maximum of 20% and further more preferably a maximum of 10% of its unstretched length. This permits the absorbent body to be applied and fixed to the material web in the stretched state so that a gathering and thereby an elastification of the absorbent body is obtained as a result of the subsequent elastic contraction of the material web.

As a further development of this inventive idea, the material web has regions of different elasticity, i.e. more preferably regions of a different modulus of elasticity. More preferably it is conceivable and advantageous when at least sections of material web in the region in which the absorbent body is positioned are formed with a lower modulus of elasticity than regions which are arranged outside the absorbent body contour.

Protection is also claimed for a disposable incontinence diaper, which has a textile outer, skin and an absorbent body joined with said outer skin during manufacture and which can be manufactured by the method according to the invention.

According to a further embodiment of the method of the invention, a further aperture is advantageously formed in the material web during execution of the textile procedure and between the apertures forming the leg apertures and is then overlaid by the absorbent body. In this manner, an aperture is formed below the absorbent body in the textile outer skin of the disposable incontinence diaper being manufactured. This is accompanied by a material saving but also has further advantages. This opening is then overlaid by the absorbent body that extends on the inside preferentially through the full circumference of the aperture. For example it would also be conceivable to provide a material reinforcement on the remaining relatively thin bridges between the leg apertures and this central opening. Reinforcing means for example can be incorporated here while carrying out the textile method. However, the reinforcement can also simply be achieved through a material accumulation of the threads in these bridge regions.

In addition, protection is claimed for a disposable incontinence diaper having such an aperture in the textile outer skin in the crotch region.

Further features, details and advantages of the invention result from the subsequent patent claims and the drawings as well as description of preferred embodiments of the method according to the invention.

The drawing shows:

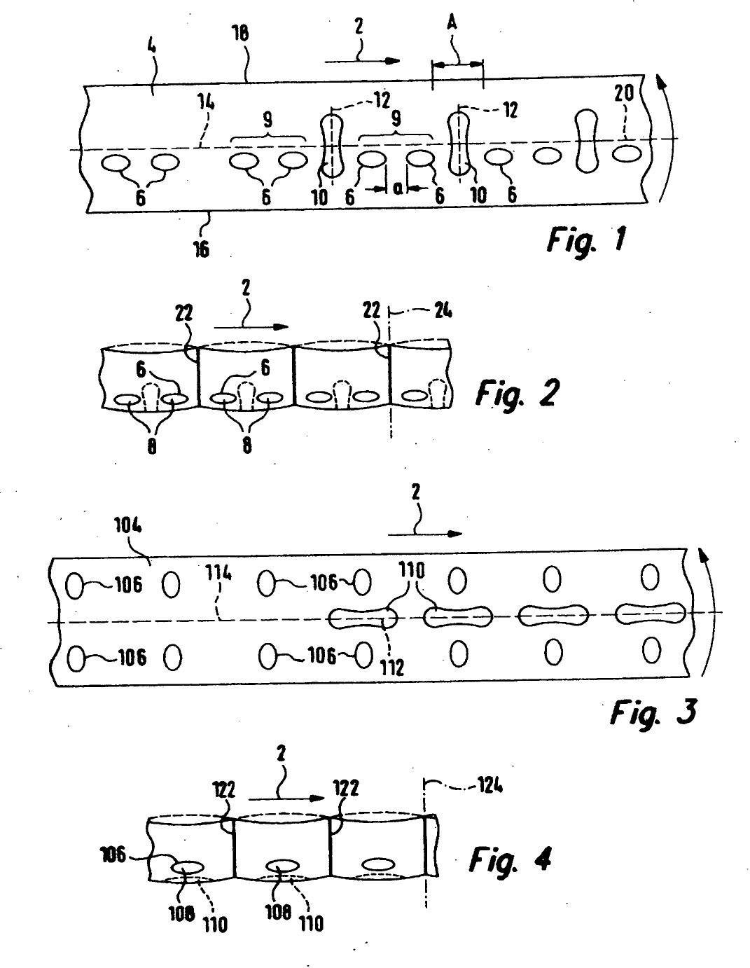

FIG. 1 a schematic view of a first version of the method configuration;

FIG. 2 a plan view of the material web folded onto itself according to the first method version;

FIG. 3 a schematic view of a second version of the method configuration according to the invention;

FIG. 4 a plan view of the material web folded onto itself according to the second method version;

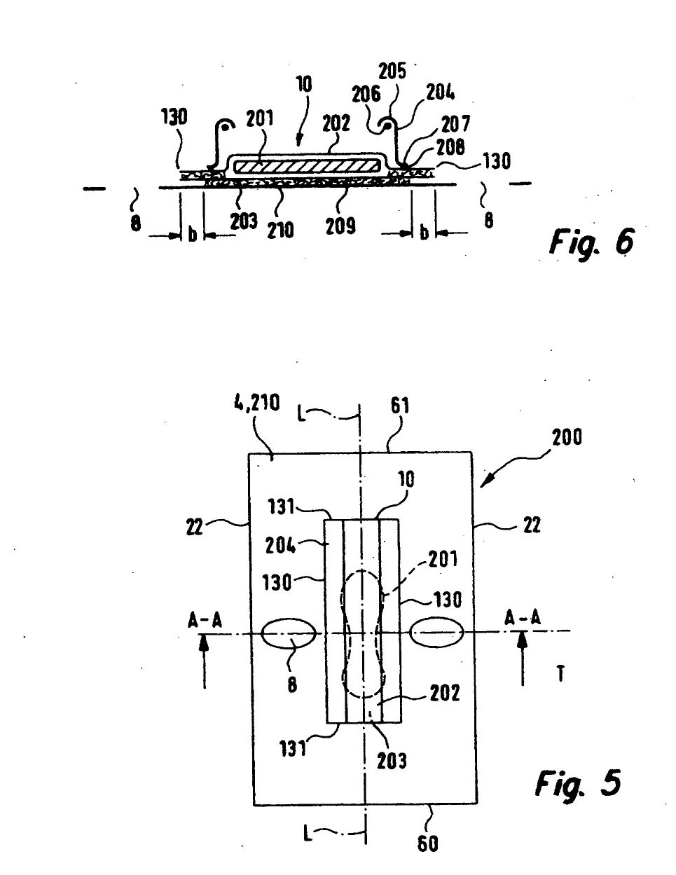

FIG. 5 a schematic plan view of the side of a diaper facing the body manufactured according to the first method version with re-opened lateral seam and folded open;

FIG. 6 a sectional view of the diaper according to FIG. 5;

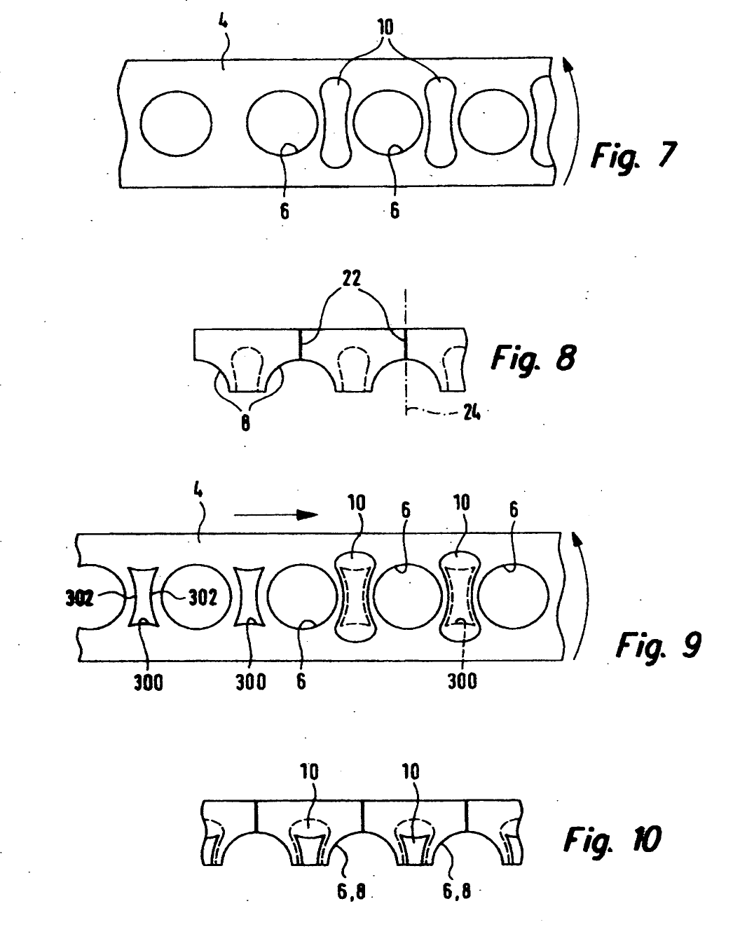

FIG. 7 a schematic view of a further embodiment of the first version of the method configuration according to the invention;

FIG. 8 a plan view of the material web folded onto itself according to the embodiment of FIG. 7;

FIG. 9 a schematic view of a further embodiment of the first version of the method according to the invention and

FIG. 10 a plan view of the material web folded onto itself according to the embodiment of FIG. 9.

FIG. 1 illustrates a first version of a method for the manufacture of a multiplicity of elastic, body fluid-absorbing disposable incontinence diapers in closed form (pants form) i.e. of disposable diapers. Towards this end, a material web 4, which is continuous in a longitudinal direction 2, is initially manufactured in a textile method i.e., for example, through weaving or knitting. During the course of carrying out the textile method, i.e. during the course of the manufacture of the continuous material web 4 which is at least elastic in longitudinal direction 2, apertures 6 which follow one another in longitudinal direction 2 and at a given distance from one another are formed in the material web, which constitute leg apertures 8 of the diapers being manufactured. In the exemplary case shown, the distance A between groups 9 of two apertures 6 is greater than the distance a between the apertures 6 within a group 9.

The material web 4 formed in this way is fed in the longitudinal direction 2 (production direction) to a diaper manufacturing machine either immediately following its continuous manufacture or for example from a reel. In addition, absorbent bodies 10 are provided and fed to the manufacturing machine and applied to the material web between each of the already mentioned groups 9 of two apertures 6. The respective absorbent body 10 is thus arranged on the material web 4 in the region of the greater distance A, in the exemplary case shown here, between pairs of apertures 6 such that its longitudinal direction 12 is oriented transverse to the longitudinal direction 2 i.e. to the production direction. In the case shown, it is also noticeable that the apertures 6 are formed outside a longitudinal center line 14 of the material web 4, i.e. transverse to the longitudinal direction 2, and are closer to one longitudinal edge 16 than to the other longitudinal edge 18 of the material web 4.

The absorbent bodies 10 are permanently fixed to the material web 4 immediately after application to the material web 4 so that the absorbent body 10 does separate from the material web 4 during later use. Towards this end, any bonding methods and any primers, adhesives, hot melt adhesives or the like can be used.

Following this, the material web 4 packed with absorbent bodies 10 is folded onto itself about an axis 20 situated in the longitudinal direction which, in the present case, is in alignment with the longitudinal center line 14, so that the view shown in FIG. 2 is obtained, wherein the absorbent bodies 10 are arranged located on the inside. In this folded state, the material web 4 is fixed onto itself transverse to the longitudinal direction 2, i.e. a joint of the sections of the material web 4 folded onto themselves transverse to the longitudinal direction 2 is established, namely in the regions between apertures 6 between which no absorbent body is located. This results in joining regions 22 which extend transverse to the longitudinal direction of a preferred extension in the region of several millimeters, more preferably 2 to 12 mm in the longitudinal direction 2. The joining regions 22 can be formed over the full area or intermittently, i.e. grid-like. To separate the diapers to be manufactured according to the invention, a separating cut, which is indicated by the dash-dotted line 24, is performed in the joining regions 22 and along the joining regions 22, i.e. transverse to the longitudinal direction 2, so that individual diapers result.

The further version of the method according to the invention shown in FIGS. 3 and 4 differs from the version shown in FIGS. 1 and 2 in that the diapers are manufactured with longitudinal conveyance. Towards this end, a material web 104 is manufactured having leg apertures 106 formed in the material web 104 in adjacent pairs transverse to the longitudinal direction 2 of the material web 104. This therefore results in two parallel rows of apertures 106 which follow one another in longitudinal direction 2. One absorbent body 110 is arranged between each pair of apertures 106 (see FIG. 3), wherein the longitudinal direction 112 of the absorbent body is in alignment with the longitudinal direction 2 and the longitudinal center line 114 of the material web 104.

Following fixation of the absorbent bodies 110 on the material web 104, the material web 104 is folded onto itself along its longitudinal center line 114 so that the arrangement shown in FIG. 4 is obtained. In the plan view of FIG. 4, a leg aperture 108 of each diaper to be manufactured is visible. Again, the material web 104 is fixed onto itself at joining regions 122 that extend across the longitudinal direction 2 and the diapers to be manufactured are separated through a separating cut 124.

FIG. 5 schematically shows a disposable incontinence diaper 200 (not to scale) in a view of the side facing the body which was manufactured according to the first method version (FIGS. 1, 2), wherein the joining regions 22 forming the lateral seam of the diaper are cut open and the diaper is folded open. The diaper has a longitudinal axis L and a transverse axis T. Here, the transverse axis T corresponds to the longitudinal axis 20 (FIG. 1), on which the material web 4 was folded. Visible is the outer skin 210 which is elastic in the direction of the transverse axis (T) formed from the material web 4 which forms the chassis of the diaper, with a front waist edge 60 and a rear waist edge 61 as well as leg apertures 8. FIG. 6 shows a sectional view along A-A of FIG. 5. The absorbent body 10 has longitudinal edge regions 130 and transverse edge regions 131 and comprises an absorbent core 201 formed of super-absorbent material and cellulose fluff which is overlaid by a liquid-permeable top sheet 202 and backed by a liquid-impermeable yet breathable back sheet 203. Band elements 204 which extend in longitudinal direction and which stand up at least in the region of the leg apertures 8 are provided on both sides of the absorbent body 10 at the distal ends 205 of which elastification means 206 are fixed in a preloaded manner. The proximal ends 207 of the band elements are fixed to the top sheet 202 using a hot melt adhesive 208. Top sheet 202 and back sheet 203 are likewise joined in a liquid-tight manner by way of a hot melt adhesive and outside the contour of the absorbent core 201.

The absorbent body is fixed to the textile skin 210 of the diaper 200, which is elastic in transverse direction, by means of a hot melt adhesive 209 and over approximately 85% of the side of its back sheet 203, facing away from the body. Fixing here only takes place in a central region. The unfixed outermost lateral edge regions 130 and transverse edge regions 131 of the absorbent body 10 remain unfixed over a width of b=10 mm, i.e. in the present case, free of adhesive. The unfixed longitudinal edges stand up slightly (not shown in FIGS. 5,6) caused by the preload effect of the elastification means 206 of the band elements 204, i.e. are raised in the direction of the diaper wearer during use, so that an additional lateral leakage bulkhead can be formed next to the band elements 204.

FIGS. 7 and 8 explain a further embodiment of the first version of the method according to the invention which differs from the method shown in FIGS. 1 and 2 in that a respective absorbent body 10 is not applied onto the web between each groups of two apertures but in a manner alternating with the apertures 6 in the material web 4. An absorbent body 10 is arranged between all successive apertures 6 which form leg apertures 8 of the disposable diapers to be manufactured. During separation in the region of the joining regions 22, a respective separating cut (indicated by the dash-dotted line 24 in FIG. 8) passes through a respective opening 6 or 8.

FIGS. 9 and 10 explain a further embodiment of the first version of the method according to the invention. As with FIGS. 7 and 8, an alternating arrangement of aperture 6 and absorbent body 10 is again formed during the course of manufacture. However, in addition to the apertures 6, which form leg apertures 8 of the disposable diapers being manufactured, a further central aperture 300, which is not round in the case shown, is formed in the crotch region of the material web 4. Like the apertures 6, the apertures 300 are formed during the course of carrying out the textile method for manufacture of the material web 4. During the course of this, two relatively narrow bridge-like regions 302 result in the crotch area between the apertures 6 or later leg apertures 8 of the disposable diaper being manufactured, which border an aperture 6 and the further central opening 300. These bridge-shaped regions 302 can be reinforced relative to the remaining regions of the material web 4 during the course of manufacture of the material web 4. This is possible, for example, by working-in reinforcement threads or merely by a concentration of threads or material. Similarly, the edge regions of the material web 4 which border the openings 6 could be formed in a reinforced manner. This measure is not restricted to the present embodiment according to FIGS. 9 and 10 but could also be realized with the remaining method versions shown.

As is evident from FIG. 9, the further central aperture 300 described in this way is overlaid by an absorbent body 10 or overlaid from the inside. Here, the absorbent body 10 is formed so that it covers the aperture 300 preferably completely, i.e. on all sides.

1-39. (canceled)

40. A method for the manufacture of a multiplicity of elastic disposable incontinence diapers in closed form (pants form) for absorbing body fluids, the method comprising the steps of:
a) manufacturing, through a textile method, an elastic material web that is continuous in a longitudinal direction and bounded by a first and a second longitudinal edge;
b) forming, during step a), successive apertures in the material web in a longitudinal direction thereof and at given distances from one another;
c) feeding the material web in the longitudinal direction;
d) supplying absorbent bodies to the material web;
e) consecutively disposing the absorbent bodies on the material web and fixing each absorbent body on the material web between pairs of apertures;
f) folding the material web onto itself about an axis disposed in the longitudinal direction;
g) fixing the material web, folded onto itself, along a direction transverse to the longitudinal direction; and
h) separating the disposable incontinence diapers by severing the material web in predetermined sections transverse to the longitudinal direction.

41. The method of claim 40, wherein the apertures form leg apertures of the disposable incontinence diaper disposed, one after the other, in the longitudinal direction of the material web.

42. The method of claim 40, wherein the apertures form leg apertures of a disposable incontinence diaper disposed next to each other in pairs, transverse to the longitudinal direction of the material web.

43. The method of claim 41, wherein the absorbent bodies are arranged between successive groups of two apertures, one after the other, in the longitudinal direction.

44. The method of claim 41, wherein an absorbent body is arranged between all apertures in the material web which form leg apertures.

45. The method of claim 40, wherein non-woven materials are used for manufacturing the absorbent bodies.

46. The method of claim 40, wherein a centrally arranged absorption core is used for the absorbent body which is overlaid by a liquid-permeable top sheet and backed by a liquid-impermeable back sheet.

47. The method of claim 40, further comprising standing-up band elements disposed on both sides of the absorbent body and extending in a longitudinal direction of the absorbent body to effect lateral leakage protection.

48. The method of claim 40, wherein the absorbent body is fixed to the material web only within an area not exceeding 90%, 80%, or 60% thereof.

49. The method of claim 40, wherein the absorbent body Is fixed to the material web only with a central region thereof.

50. The method of claim 40, wherein the absorbent body is fixed to the material web forming unfixed, free longitudinal edge regions and/or unfixed free transverse edge regions.

51. The method of claim 50, wherein the unfixed longitudinal edge regions and/or transverse edge regions have an extension, measured as a shortest distance from an absorbent body edge to the start of fixation, of at least 3 mm, at least 8 mm, or at least 15 mm.

52. The method of claim 40, wherein the absorbent body is fixed to the material web over an entire area thereof.

53. The method of claim 40, wherein at least 30% by weight, a maximum of 90% by weight, a maximum of 80% by weight, or a maximum of 70% by weight of super-absorbent polymer materials (SAP), based on the mass of the absorption core, are used during manufacture of the absorbent body.

54. The method of claim 40, wherein the axis disposed in the longitudinal direction about which the material web is folded onto itself is oriented along a longitudinal center line and has a same distance from the first and second longitudinal edges.

55. The method of claim 40, wherein cotton is used to manufacture the material web.

56. The method of claim 40, wherein a thermoplastic material is used to manufacture the material web.

57. The method of claim 40, wherein fixation of the material web, folded onto itself, is carried out along a direction transverse to the longitudinal direction through thermal welding or ultrasonic welding.

58. The method of claim 40, wherein fixation of the material web, folded onto itself, along a direction transverse to the longitudinal direction is carried out using an adhesion-promoting binder.

59. The method of claim 40, wherein a lateral seam is provided for fixation of the material web, folded onto itself, along a direction transverse to the longitudinal direction.

60. The method of claim 40, wherein the material web, fixed onto itself, can be torn open again along a fixation.

61. The method of claim 40, wherein the material web, fixed onto itself, can be opened and closed again along a fixation.

62. The method of claim 40, wherein a breathable back sheet is used for manufacture of the absorbent body.

63. The method of claim 40, wherein an elastic back sheet is used to manufacture the absorbent body.

64. The method of claim 40, wherein the elastic material web is formed with a breathability that is at least twice that of a back sheet of the absorbent body.

65. The method of claim 40, wherein following step h), the disposable incontinence diaper is turned inside out using a reversing device.

66. The method of claim 41, wherein the apertures are formed on a longitudinal center line with a same distance to the first and second longitudinal edges.

67. The method of claim 41, wherein the apertures are formed off center and closer to one of the first and second longitudinal edges.

68. The method of claim 40, wherein feeding of the material web takes place during application of the absorbent bodies at a web speed of at least 50 m/min or of at least 100 m/min.

69. The method of claim 40, wherein the material web is fed in a stretched state.

70. The method of claim 69, wherein the material web is fed stretched by a maximum of 20% or by a maximum of 10% of an unstretched length thereof.

71. The method of claim 69, wherein the absorbent body is applied to and fixed with the material web in a stretched state.

72. The method of claim 40, wherein the material web has regions of differing elasticity.

73. The method of claim 40, wherein, upon carrying out the textile method, a further aperture is formed in the material web between two apertures forming leg apertures and is then overlaid by the absorbent body.

74. A body fluid absorbent disposable incontinence diaper in closed form manufactured by the method of claim 40 and having a textile elastic outer skin with leg apertures and an absorbent body joined, during manufacturer, with said outer skin.

75. A body fluid absorbent disposable incontinence diaper in closed form manufactured by the method of claim 40, having a textile elastic outer skin, leg apertures, and an absorbent body joined during manufacturer with said outer skin, wherein the leg apertures are formed during manufacture of a material web forming the outer skin in a textile method, wherein a further central aperture is provided in the textile outer skin between the leg apertures and is overlaid from the inside by the absorbent body.

76. A disposable incontinence diaper of claim 75, wherein the further central aperture is formed corresponding to an outer circumferential contour of the absorbent body.

77. The disposable incontinence diaper of claim 74, wherein the absorbent body comprises a liquid-permeable top sheet, a liquid-impermeable back sheet, and an absorption core arranged in-between, which form an integral unit and are applied as such to the inner side of the outer skin.
